(19) United States
(12) Patent Application Publication
(10) Pub. No.: US 2023/0069730 A1
(43) Pub. Date: Mar. 2, 2023

(54) FLOW BLOCKING CATHETER

(71) Applicant: MICROPORT NEUROTECH (SHANGHAI) CO., LTD., Shanghai (CN)

(72) Inventors: Yuxi CUN, Shanghai (CN); Yunyun LIU, Shanghai (CN); Heng LIN, Shanghai (CN); Yumei LIU, Shanghai (CN)

(21) Appl. No.: 17/460,869

(22) Filed: Aug. 30, 2021

(51) Int. Cl. A61M 25/10; A61M 25/00; A61L 29/08

(52) U.S. Cl. CPC A61M 25/104; A61M 25/10185; A61M 25/0012; A61M 25/0045; A61L 29/085

(57) ABSTRACT

A flow blocking catheter including an inner tube, an outer tube and a flow blocking member is provided. The flow blocking member has one end attached to an outer circumference of the inner tube and the other end attached to a distal end of the outer tube. The flow blocking member is configured to expand as the outer tube moves toward a distal end of the inner tube and to collapse as the outer tube moves away from the distal end of the inner tube. In this way, expansion of the flow blocking member is able to be controlled simply by pushing/retracting the outer or inner tube to offer a fast shifting between different configurations. The flow blocking member is able to occlude blood flow with a controllably expansion to lower stimulation to the wall of the blood vessel and avoid the easy bursting of the balloon.

### TECHNICAL FIELD

The present application relates to the field of medical instruments and, in particular, to a flow blocking catheter.

### BACKGROUND

Strokes, mainly caused by blood clots in cerebral blood vessels, are a common medical condition that seriously threatens human health, which is also the third leading cause of death worldwide and the leading cause of long-term disability in adults. In the current clinical practice, treatments of directly sucking the thrombus with an aspiration catheter or removing the thrombus with the assistance of a stent are used to eliminate the thrombus to achieve recanalization of the blood vessel. After the aspiration catheter reaches the thrombus location along the blood vessel, a negative pressure is applied at its proximal end to suck the clot into the aspiration catheter or onto the opening of aspiration catheter, followed by slow retraction of the clot into a guide catheter. As a result, the blood vessel recovers back to its normal hemodynamic condition. The thrombectomy stent is required to cross over the clot, trap the clot within meshes of the stent and then retract back into the support catheter, so as to recanalize the blood vessel. After the stent is retracted back into the support catheter, the support catheter together with the stent and blood clot trapped in the stent, is in turn withdrawn into the guide catheter. However, during the thrombus removal process, the fragment clots often fall off and are rushed to the distal blood vessel due to the impact of proximal blood flow, or during the operation of the aspiration catheter or the delivery of thrombectomy stent into the interventional instrument (the guide or support catheter) after the successful capture of clots, the clots break up to create clot fragments that are rushed to the distal blood vessel by the blood flow to cause secondary blocking, which results in the failure of operation and may even threaten the patient's life in severe cases. For example, the possibility of percutaneous coronary intervention (PCI) caused myocardial necrosis reaches as high as 16%-39%, and most of these cases have been found to be attributable to escape of clots into distal blood vessels during the intervention operation. In order to solve the problems caused by clot fragmentation, the balloon guide catheter has been adopted commonly in prior art to facilitate the thrombus removal operation by temporarily occluding the blood flow.

Typically, during the operation, after a thrombus removal device is delivered to a target site with the assistance of a balloon guide catheter (i.e., an aspiration or support catheter passes through a lumen of the balloon guide catheter to reach the target site), the balloon is expanded against the blood vessel wall by injecting a radiopaque fluid in the balloon so as to temporarily occlude blood flow in the vessel. Moreover, after the blood clot has been taken into a lumen of the aspiration or support catheter, the balloon is contracted, followed by withdrawal of the balloon guide catheter. In this way, the blood clot is taken out of the patient's body to achieve the effect of blood flow reconstruction.

However, in existing balloon guide catheters, the balloon is typically provided on the outside of an outer tube. As the balloon has a certain thickness itself and given that an outer diameter of the catheter must be designed to be not too large to ensure its smooth passage in blood vessels, the catheter has to assume a very small inner diameter, making it impossible to be fitted with a wide-lumen aspiration or support catheter. This therefore makes it unable to treat large-size thrombi. Moreover, for balloon guide catheter, since it is necessary to fill the balloon with the radiopaque or other liquid to make the balloon bulge and attach to the blood vessel wall for blocking the blood flow, it may take some time to achieve a complete blood flow blocking effect. It may also be the case for the withdrawal of the balloon guide catheter by drawing out the radiopaque fluid. This not only prolongs the surgical time, but may also lead to ischemia or even necrosis of the tissue due to an excessively long blood flow blocking time. This may also results in a risk of blood vessel damage from over-expansion or bursting of the balloon. More importantly, during surgery, if it is found that the balloon is dilated at an improper location, the radiopaque fluid has to be completely discharged before the balloon can be relocated and re-expanded. This not only takes much more time but also increases risk of bursting of the balloon to cause secondary damage to the blood vessel due to the repeated dilation. Further, the pressure exerted by the dilated balloon tends to stimulate the wall of the cerebral blood vessel and thus cause various complications during the surgical procedure. All these shortcomings limit the benefits of using balloons in thrombus removal procedures, increase complexity of such procedures and expose the patients to high risk.

### SUMMARY

It is an object of the present application to provide a flow blocking catheter to overcome the problems of slow flow blocking, low safety and reliability, poor reproducibility and small catheter lumen of existing guide catheters that are brought by the use of balloon for blood flow blocking.

To solve the above problem, present application provides a flow blocking catheter comprising:

an inner tube;

an outer tube movably sleeved on the exterior of the inner tube; and

a flow blocking member having one end attached to an outer circumference of the inner tube and the other end attached to a distal end of the outer tube, wherein the flow blocking member is configured to expand as the outer tube moves toward a distal end of the inner tube and to collapse as the outer tube moves away from the distal end of the inner tube.

Optionally, in the flow blocking catheter, the flow blocking member comprises a support frame having opposing ends thereof attached to the inner tube and the outer tube respectively, wherein the support frame is configured to expand when axially compressed and collapse when axially pulled.

Optionally, in the flow blocking catheter, the flow blocking member further comprises a flow blocking membrane attached to the support frame.

Optionally, in the flow blocking catheter, the distal end of the inner tube comprises an expanded section that has an outer circumferential size greater than that of rest portion of the inner tube, wherein the flow blocking member is connected to the expanded section at one end and to the distal end of the outer tube at the other end.

Optionally, in the flow blocking catheter, the outer circumference of the expanded section is sized to fit with an outer circumferential size of the outer tube.

Optionally, in the flow blocking catheter, the flow blocking member further comprises an isodiametric section that has an equal outer circumferential size along an axial direction of the flow blocking member in an expanded configuration.

Optionally, the flow blocking catheter further comprises a control valve configured to drive relative movements between the inner and outer tubes.

Optionally, in the flow blocking catheter, the control valve comprises a control valve body and a control slider configured to be axially slidable, wherein: the control valve body is coupled to a proximal end of the inner tube with the control slider being coupled to a proximal end of the outer tube; or the control valve body is coupled to the proximal end of the outer tube with the control slider being coupled to a proximal end of the inner tube.

Optionally, in the flow blocking catheter, both or either of the inner tube and the outer tube is a single-layered tube made of macromolecular material.

Optionally, in the flow blocking catheter, both or either of the inner tube and the outer tube has a structure comprising at least two layers, in which both or either of a first layer and a second layer from inside to outside is made of macromolecular.

Optionally, in the flow blocking catheter, both or either of the inner tube and the outer tube has a structure comprising at least two layers, in which a second layer from inside to outside comprises one or more selected from the group consisting of braided structure, coil, and cut hypotube.

Optionally, in the flow blocking catheter, each of the inner and outer tubes has a triple-layered structure.

Optionally, in the flow blocking catheter, the inner tube comprises a first radiopaque ring disposed at a distal end of the inner tube.

Optionally, in the flow blocking catheter, the inner tube further comprises a second radiopaque ring disposed at a location of the inner tube where the flow blocking member is attached to the inner tube.

Optionally, in the flow blocking catheter, the outer tube further comprises a third radiopaque ring disposed at a location of the outer tube where the flow blocking member is attached to the outer tube.

Optionally, in the flow blocking catheter, the flow blocking member comprises at least one selected from the group consisting of mesh structure, open-loop structure and spiral structure, and the flow blocking member is fabricated by braiding, winding or cutting.

Optionally, in the flow blocking catheter, the mesh structure is braided from 1-64 filaments. The filament is at least one selected from the group consisting of regular filament, radiopaque filament and composite filament. The regular filaments is made of at least one selected from the group consisting of nickel-titanium alloy, cobalt-chromium alloy, stainless steel and macromolecular material. The radiopaque filaments is made of at least one selected from the group consisting of radiopaque metal, alloy of radiopaque metals and macromolecular material containing a radiopaque agent. The composite filament is formed by a radiopaque core filament combined with a regular filament.

In summary, the flow blocking catheter of the present application comprises an inner tube, an outer tube movably sleeved on the exterior of the inner tube; and a flow blocking member having one end attached to an outer circumference of the inner tube and the other end attached to a distal end of the outer tube. The flow blocking member is configured to expand as the outer tube moves toward a distal end of the inner tube and to collapse as the outer tube moves away from the distal end of the inner tube.

With this configuration, expansion of the flow blocking member is able to be controlled simply by pushing/retracting the outer or inner tube, which allows to achieve a fast shifting between different configurations, relocatability during a surgical procedure, and simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or passage of large instruments.

### BRIEF DESCRIPTION OF THE DRAWINGS

Those of ordinary skill in the art would appreciate that the appended figures are presented merely to enable a better understanding of the present application rather than limit the scope thereof in any sense. In the figures,

FIG. 1 is a schematic diagram of a flow blocking catheter according to a preferred embodiment of the present application;

FIG. 2 is a schematic diagram of a flow blocking member in a collapsed configuration according to a preferred embodiment of the present invention;

FIG. 3 is a schematic diagram of a flow blocking member in an expanded configuration according to a preferred embodiment of the present invention;

FIG. 4 is a schematic diagram of a control valve according to a preferred embodiment of the present application;

FIG. 5 is a schematic cross-sectional view of an inner tube according to a preferred embodiment of the present application;

FIG. 6 is a schematic cross-sectional view of a flow blocking catheter according to a preferred embodiment of the present application;

FIG. 7 is a schematic diagram of a flow blocking catheter provided with an expanded section according to a preferred embodiment of the present application;

FIG. 8 is a schematic diagram of a flow blocking member provided with an isodiametric section according to a preferred embodiment of the present application;

FIG. 9 is a schematic diagram of a braided structure of a flow blocking member according to a preferred embodiment of the present application; and

FIGS. 10a to 10g schematically illustrate various mesh openings of support frames according to preferred embodiments of the present application.

In the figures,

100, inner tube; 101, first layer; 102, second layer; 103, third layer; 104, adhesive; 110, expanded section; 120, first radiopaque ring;

200, outer tube; 210, distal end of the outer tube; 220, stress dispersion tube;

300, flow blocking member; 310, first end; 320, second end; 330, radiopaque filament; 340, mesh opening; 350, isodiametric section;

400, control valve; 410, control valve body; 420, control slider; 430, sliding slot; 440, catheter insertion opening; 500, securing film.

### DETAILED DESCRIPTION

To make objects, advantages and features of the present application more apparent, present application is described in detail by the particular embodiments made in conjunction with the accompanying drawings. Note that the figures are provided in a very simplified form not necessarily drawn to exact scale, with the only intention to facilitate convenience and clarity in explaining the present application. In addition, structures shown in the figures are usually part of actual structures. In particular, as the figures tend to have distinct emphases, they are often drawn to different scales.

As used in present specification, the meaning of “a,” “an,” and “the” include singular and plural references, unless the context clearly dictates otherwise. As used in present specification and appended claims, the term “or” generally includes the meaning of “and/or”, unless the context clearly dictates otherwise. Additionally, the terms “proximal” and “distal” are generally used to refer to an end close to an operator and an end close to a lesion site in a patient, respectively. Further, the terms “one end” and “the other end”, or “proximal end” and “distal end”, are generally used to refer to two opposing portions including not only the endpoints.

The core idea of the present application is to provide a flow blocking catheter to overcome the problems of slow flow blocking, low safety and reliability, poor reproducibility and small catheter lumen of existing guide catheters that are brought by the use of balloon for blood flow blocking. The flow blocking catheter comprises an inner tube, an outer tube movably sleeved on the exterior of the inner tube; and a flow blocking member having one end attached to an outer circumference of the inner tube and the other end attached to a distal end of the outer tube. The flow blocking member is configured to expand as the outer tube moves toward a distal end of the inner tube and to collapse as the outer tube moves away from the distal end of the inner tube. With this configuration, expansion of the flow blocking member is able to be controlled simply by pushing/retracting the outer or inner tube, which allows to achieve a fast shifting between different configurations, a few influence on tissue blood supply, relocatability during a surgical procedure, and simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or instruments.

In the following description, reference is made to FIGS. 1 to 10g.

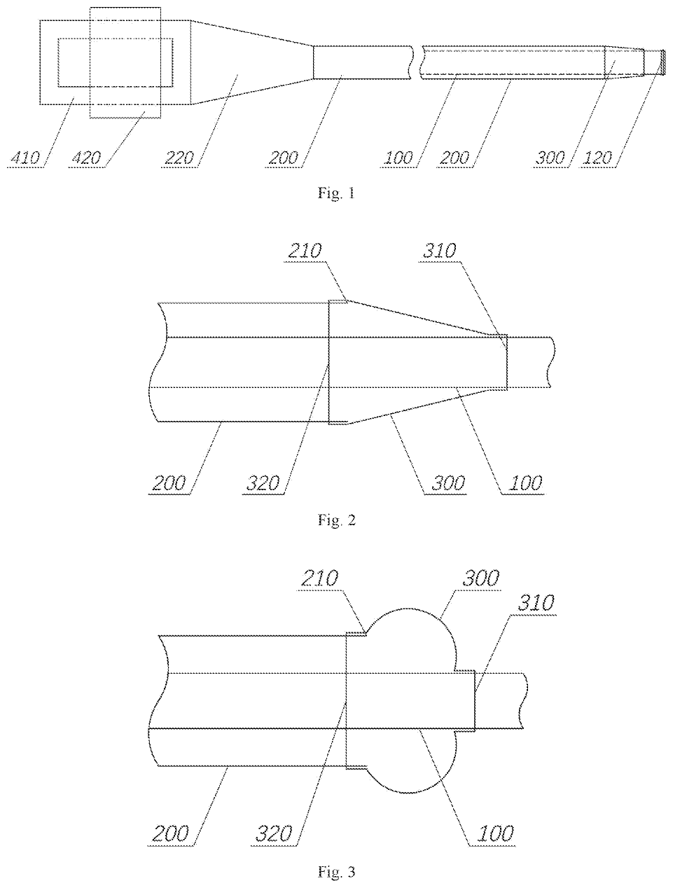

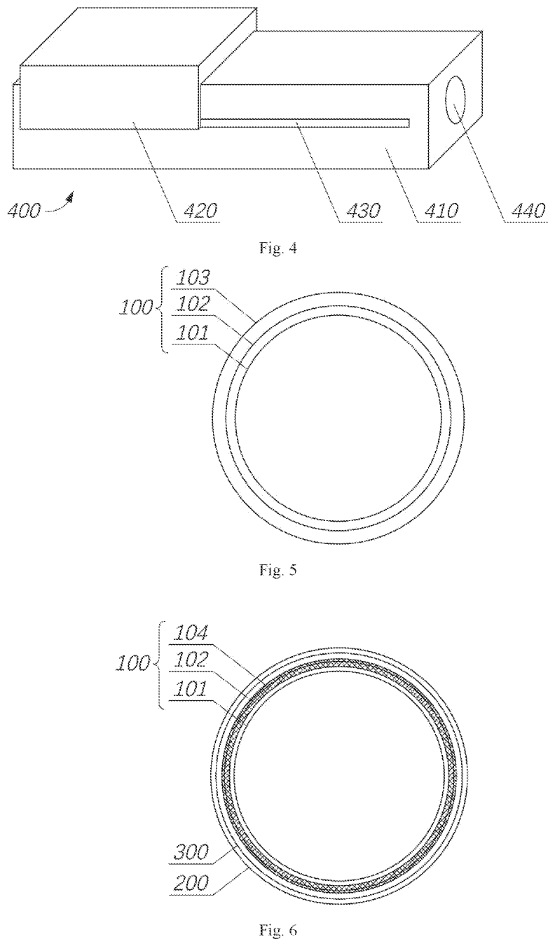

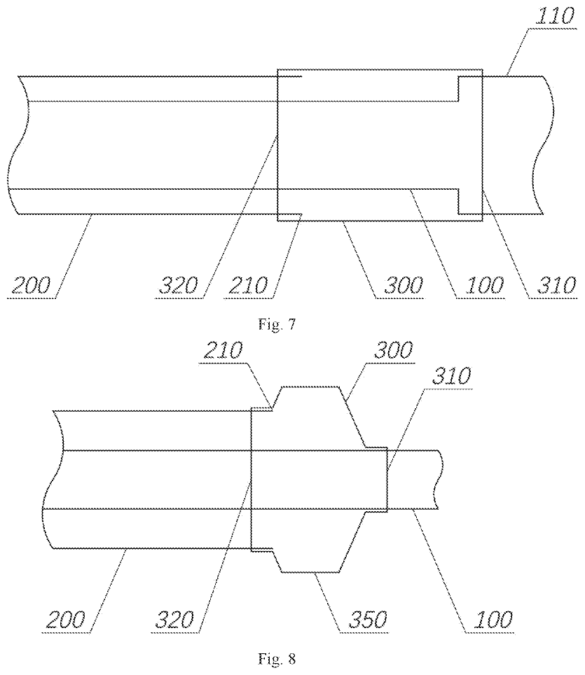

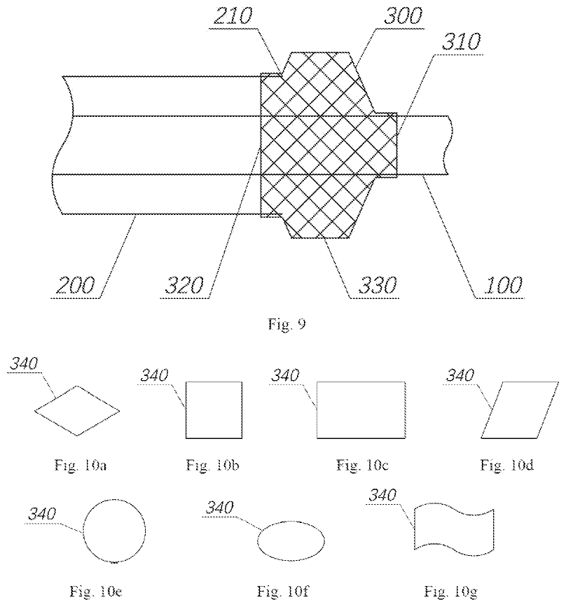

Please refer to FIG. 1 to FIG. 10g, in which FIG. 1 is a schematic diagram of a flow blocking catheter according to a preferred embodiment of the present application; FIG. 2 is a schematic diagram of a flow blocking member in a collapsed configuration according to a preferred embodiment of the present invention; FIG. 3 is a schematic diagram of a flow blocking member in an expanded configuration according to a preferred embodiment of the present invention; FIG. 4 is a schematic diagram of a control valve according to a preferred embodiment of the present application; FIG. 5 is a schematic cross-sectional view of an inner tube according to a preferred embodiment of the present application; FIG. 6 is a schematic cross-sectional view of a flow blocking catheter according to a preferred embodiment of the present application; FIG. 7 is a schematic diagram of a flow blocking catheter provided with an expanded section according to a preferred embodiment of the present application; FIG. 8 is a schematic diagram of a flow blocking member provided with an isodiametric section according to a preferred embodiment of the present application; FIG. 9 is a schematic diagram of a braided structure of a flow blocking member according to a preferred embodiment of the present application; and FIGS. 10a to 10g schematically illustrate various mesh openings of support frames according to preferred embodiments of the present application.

As shown in FIGS. 1 to 3, a flow blocking catheter according to an embodiment includes an inner tube 100, an outer tube 200 and a flow blocking member 300. The flow blocking member 300 is sleeved on the exterior of the inner tube 100. One end (the first end 310) of the flow blocking member 300 is attached (e.g., by adhesive bonding, welding or a securing film) to an outer circumference of the inner tube 100 and the other end (the second end 320) of the flow blocking member 300 is attached (e.g., by adhesive bonding, welding or a securing film) to the distal end of the outer tube 200 (distal end 210 of the outer tube). The flow blocking member 300 is configured to expand (i.e., bulge radially) as the outer tube 200 moves toward a distal end of the inner tube 100 and to collapse (i.e., retract and recover radially) as the outer tube 200 moves away from the distal end of the inner tube 100 (i.e., towards a proximal end of the inner tube 100). In still other embodiments, expansion and collapse of the flow blocking member 300 is able to be controlled by movements of the inner tube 100 relative to the outer tube 200. In this embodiment, the first end 310 of the flow blocking member 300 is arranged close to the distal end of the inner tube 100 so that blood blocking position is close to the location where a thrombus removal or other instrument operates, thus reducing adverse impact on blood flow in the proximal blood vessel. In alternative embodiments, the first end 310 of the flow blocking member 300 may also be arranged at the middle of the inner tube 100 or close to the proximal end of the inner tube 100.

In one exemplary embodiment, both the inner 100 and outer 200 tubes are preferred to be circular tubes and the outer tube 200 is sleeved on the inner tube 100. The difference between an outer diameter of the inner tube 100 and an inner diameter of the outer tube 200 may range from 0.0001 inch to 0.1 inch. The outer tube 200 is preferred to be a single-layered tubular member formed of, for example, one or more of a polyether-polyamide block copolymer (PEBA or Pebax), polyamide (PA) and polytetrafluoroethylene (PTFE). The inner tube 100 includes at least a single macromolecular layer made of a macromolecular material that may be one or more selected from the group consisting of PTFE, high-density polyethylene (HDPE), Pebax mixed with a friction coefficient reducing additive, and polyolefin elastomer (POE). Preferably, the inner tube 100 includes a triple-layered structure, as shown in FIG. 6, consisting of a first layer 101, a second layer 102, and a third layer 103 arranged from inside to outside. The third layer 103 may be formed of, for example, one or more of the nylon elastomer (e.g., Pebax), nylon and polyurethane (PU). The first layer 101 may be formed of, for example, one or more of PTFE, HDPE, Pebax mixed with a friction coefficient reducing additive, and POE. The second layer 102 may consist of any one of a braided structure, a coil and a cut hypotube (here, the term “hypotube” refers to any metal tube for medical use), or a combination of two or more thereof. The second layer 102 may be formed of stainless steel, nickel-titanium alloy, cobalt-chromium alloy or macromolecular wire, which can increase force transmission performance, ellipticity resistance and bending resistance of the inner tube 100 as well as reduce a force required to withdraw the flow blocking member 300. It is to be understood that materials of the layers of the inner tube 100 are not limited to the materials listed above, and those skilled in the art may also choose other functionally similar materials based on prior art. As shown in FIG. 5, in one alternative embodiment, the inner tube 100 includes only two layers, which are a first layer 101 and a second layer 102 covered on the first layer. In this case, the first layer 101 may be essentially a macromolecular layer formed of one or more of PTFE, HDPE, Pebax mixed with a friction coefficient reducing additive, and POE. The second layer 102 may be essentially a metallic layer consisting of, for example, any one of a braided structure, a coil and a cut hypotube, or a combination of two or more thereof. The second layer 102 may be formed of for example, stainless steel, a nickel-titanium alloy, a cobalt-chromium alloy or the like. Preferably, a layer of adhesive 104 may be applied onto the macromolecular layer, which penetrates into the metallic layer (i.e., part of the adhesive 104 penetrates through meshes formed in the metallic layer and adheres to the exterior of the macromolecular layer) to form a firm adhesion between the metallic and macromolecular layers, so as to improve the force transmission performance and ellipticity resistance. Of course, the outer tube 200 is not limited to being a single-layered tube in other embodiments, and it may also be implemented as a double-, triple- or more-layered structure. The specific structure of the outer tube 200 can refer to that of the inner tube 100.

Preferably, the inner tube 100 includes a first radiopaque ring 120 disposed at the distal end of the inner tube 100. In particular, the first radiopaque ring 120 may be disposed at a distal end of the second layer 102 of the inner tube 100. More preferably, the inner tube 100 further includes a second radiopaque ring (not shown) disposed at a location of the inner tube 100 where the flow blocking member 300 is attached to the inner tube 100. Further, the flow blocking member 300 further includes a third radiopaque ring (not shown) disposed at a location of the outer tube 200 where the flow blocking member 300 is attached to the outer tube 200. Optionally, examples of materials of the first 120, second and third radiopaque rings may include, but are not limited to, platinum, iridium, tantalum, noble metal alloys and macromolecular materials containing radiopaque agents. Arranging the three radiopaque rings helps the operator locate the inner tube 100 during a surgical procedure. It is to be understood that the first radiopaque ring 120 is located at the distal end of the inner tube 100, but it is not intended to limit that the first radiopaque ring 120 can only be located at the distal end face of the inner tube 100, which can be located in an area close to the distal end of the inner tube 100. While the above embodiment exemplifies the positions of the three radiopaque rings, it is not intended to limit that the three radiopaque rings must be provided at the same time, and those skilled in the art may select to provide any one or two of them according to the actual circumstances.

Preferably, the flow blocking member 300 includes a support frame, and a flow blocking membrane attached to the support frame. The opposing ends of the support frame are attached to the inner tube 100 and outer tube 200 respectively. The support frame is configured to expand (i.e., bulge radially) when axially compressed and to collapse (i.e., retract and recover radially) when axially pulled. In one example, the support frame is a tubular member that is able to switch between a collapsed configuration and an expanded configuration. It is to be understood that the support frame is not limited to switch only between the collapsed configuration and the expanded configuration. In some cases, it may also assume an intermediate configuration between the collapsed and expanded configurations (i.e., a semi-expanded or partially-expanded configuration). The support frame may be formed of, for example, nickel-titanium alloy, Type 304 stainless steel, platinum-tungsten alloy, platinum-iridium alloy, cobalt-chromium alloy, radiopaque metal or the like. The support frame may be fabricated by winding, cutting or braiding. In this embodiment, the support frame includes a plurality of mesh openings 340, as shown in FIGS. 10a to 10g. The mesh opening 340 may have a rhombic (10a), square (10b), rectangular (10c), parallelogramic (10d), polygonal (not shown), circular (10e), elliptic (10f) or irregular (10g) shape, with the rhombic shape (10a) being preferred. The flow blocking membrane may be attached to either an inner surface or an outer surface of the support frame. The flow blocking membrane is preferably a macromolecular membrane formed of, for example, PU, polyethylene(PE), expanded polytetrafluoroethylene (ePTFE) or the like. It is to be understood that the support frame and the flow blocking membrane are not limited to being formed of the materials listed above, and those skilled in the art may also choose other functionally similar materials based on prior art. As shown in FIG. 9, in some embodiments, the support frame may be a mesh structure braided from 1-64 filaments. The filament may be at least one selected from the group consisting of regular filament, radiopaque filament and composite filament. The material of the regular filament may be selected as, but is not limited to, one or more of nickel-titanium alloy, cobalt-nickel alloy, stainless steel, macromolecular material and the like. The material of the radiopaque filament may be selected as, but is not limited to, radiopaque metal such as platinum, iridium, gold or tungsten, or the alloy thereof, or a macromolecular material containing a radiopaque agent of the radiopaque metal or alloy. The radiopaque filament 330 enhances radiopaque property of the flow blocking member 300, and the traceability of the flow blocking member 300 during use. In alternative embodiments, the support frame may be an open-loop structure or a spiral structure, or consist of two or more of a mesh structure, an open-loop structure and a spiral structure.

Referring to FIGS. 2 and 3, expansion state of the flow blocking member 300 (it would be appreciated that expansion state of the flow blocking member 300 is the same as that of the support frame) can be controlled by movement of the outer tube 200 along an axis of the inner tube 100. Specifically, as shown in FIG. 2, for ease of description, the point where the first end 310 of the flow blocking member 300 is attached to the inner tube 100 is referred to hereinafter as a first attachment point, and the point where the second end 320 of the flow blocking member 300 is attached to the outer tube 200 as a second attachment point. In an initial configuration of the flow blocking catheter, distance between the first attachment point and the second attachment point along the axial direction of the inner tube 100 is the largest. At this moment, the flow blocking member 300 is in a fully collapsed configuration with a maximum outer diameter that is comparable to an outer diameter of the outer tube 200. Based on the configuration of FIG. 2, the outer tube 200 is pushed distally to decrease the axial distance between the first and second attachment points, which causes the flow blocking member 300 to expand outward along its radial direction, as shown in FIG. 3 where the flow blocking member 300 is in a fully expanded configuration. When the flow blocking member 300 expands outwardly to the extent that it fits the inner diameter of the blood vessel wall, the flow blocking member 300 contacts and attaches to the blood vessel wall. The blood flow in the blood vessel is thus blocked as a layer of the flow blocking membrane is attached to the support frame of the flow blocking member 300. It is to be understood that, at this point of time, the expansion of the flow blocking member 300 adapts to the dimension of the blood vessel wall, and the flow blocking member 300 is not necessarily fully expanded (i.e., flow blocking member 300 may be in a semi-expanded or partially-expanded configuration). Preferably, the flow blocking member 300 is compliant to a certain extent, which makes it able to adapt shapes of the blood vessel walls in an expanded configuration (including the fully-, semi- or partially-expanded configuration). This arrangement is favorable to blood vessels with vulnerable walls by reducing the force applied to these blood vessel walls from the expansion of the flow blocking member 300. As a result, the flow blocking member 300 is able to lower stimulation for the wall of a cerebral blood vessel, suppress the occurrence of various complications such asvasospasm during surgery and completely avoid the risk of secondary damage to the blood vessel caused by the bursting of a balloon or a balloon bonding section.

Further, when blood flow blocking has been attained, a blood clot can be directly sucked, or captured and pulled back via the lumen of the inner tube 100 (a aspiration catheter may be deployed in the lumen of the inner tube 100 of the flow blocking catheter to suck the clot, or a support catheter may be deployed in the lumen, in which a thrombectomy stent is provided for removing the clot). As shown in FIG. 2, because of a relative small thickness of the flow blocking member 300 in the collapsed configuration, the lumen of the inner tube 100 takes up a much larger proportion of a cross-sectional area of the flow blocking catheter, when compared to the case of using a conventional balloon catheter. Therefore, for a given outer diameter, the flow blocking catheter of present application is able to be fitted with aspiration catheters or support catheters with large lumens, stents or other medical instruments for treating large blood clots, while the outer diameter of the entire flow blocking catheter is limited to ensure that the flow blocking catheter is able to pass through tortuous distal blood vessels successfully and causes a small wound to the patient.

Further, when it is necessary to change positions of the blood flow blocking by relocating or withdrawing the flow blocking catheter, the outer tube 200 may be caused to move proximally relative to the inner tube 100 (i.e., retracting the outer tube 200) until the distance between the first and second attachment points along the axial of the inner tube 100 becomes maximum. The configuration of the flow blocking member 300 shown in FIG. 3 is reversible when the outer tube 200 is retracted proximally, i.e., the flow blocking member 300 is able to be collapsed to return to the configuration shown in FIG. 2. The repeatable collapse of the flow blocking member 300 enables easy re-delivery and relocation of the flow blocking catheter. Therefore, the flow blocking catheter of this embodiment provides convenience for achieving repetitive operations, accurate location as well as convenience for achieving withdrawal from the blood vessel with the removed thrombus.

As shown in FIG. 4, the flow blocking catheter further includes a control valve 400 configured to drive movements of the outer tube 200 relative to the inner tube 100. In one embodiment, the control valve 400 includes a control valve body 410 and a control slider 420. The control valve body 410 is provided with a sliding slot 430 along its axial direction. The sliding slot 430 matches with the control slider 420 to enable the control slider 420 to slide along the sliding slot 430. Further, one end of the control valve body 410 defines a catheter insertion opening 440, through which proximal end of the inner tube 100 inserts into the control valve 400 and fixedly coupled to the control valve body 410 while the proximal end of the outer tube 200 is coupled to the control slider 420, for example, by adhesive bonding or snap-fitting. With this arrangement, movements (e.g., backward retraction or forward push) of the outer tube 200 relative to the inner tube 100 is able to be controlled by controlling sliding of the control slider 420. In this way, the control valve 400 is able to control expansion or contraction of the flow blocking member 300, thus simplifying operations involved in the surgical procedure, shortening the operation time and providing convenience for repeat operations. Optionally, proximal end of the outer tube 200 includes a stress dispersion tube 220. The stress dispersion tube 220 flares towards the proximal end. That is, the distal end of the stress dispersion tube 220 has a diameter equal to the diameter of the outer tube 200, and the proximal end of the stress dispersion tube 220 has a diameter greater than the diameter of the outer tube 200. With this arrangement, portion of the outer tube 200 configured to couple the control slider 420 has a widened diameter, and the flaring stress dispersion tube 220 helps in dispersing a drive force exerted by the control slider 420 on the outer tube 200, enabling to achieve a more reliable control of outer tube 200 by the control slider 420. In alternative embodiments, it is also possible to couple the outer tube to the control valve body 410, with the inner tube 100 being coupled to the control slider 420. Other direct or indirect coupling designs are also possible, and the present application is not limited in this regard.

Referring to FIG. 7, in one preferred embodiment, the inner tube 100 includes a expanded section 110 at the outer circumference of its distal end. The expanded section 110 has an outer circumferential size greater than that of rest portions of the inner tube 100. In this case, the blood blocking member 300 is connected to the expanded section 110 at one end (the first end 310) and to the distal end 210 of the outer tube at the other end (the second end 320). Here, the “outer circumferential size” refers to a cross-sectional perimeter of the inner tube 100 or the outer tube 200. For example, when each of the inner tube 100 and the outer tube 200 is a circular tube, the “outer circumferential size” refers to the outer diameter of the inner tube 100 or outer tube 200. Preferably, the outer circumferential size of the expanded section 110 is matched with that of the outer tube 200, and the expanded section 110 is located distally relative to the distal end of the outer tube 200. In one exemplary embodiment, the main section of the inner tube 100 has an outer diameter of 0.070-0.113 inches and a length of 70-100 mm, while the expanded section 110 has an outer diameter of 0.079-0.122 inches and a length of 1-50 mm. Preferably, an inner diameter of the outer tube 200 is slightly greater than the outer diameter of the main section of the inner tube 100, and the outer tube 200 is sleeved on the main section of the inner tube 100. The outer diameter of the outer tube 200 may be equal to that of the expanded section 110. Preferably, the axial distance between the distal end 210 of the outer tube and the expanded section 110 may be 5-50 mm. Moreover, the first attachment point between the first end 310 of the blood blocking member 300 and the expanded section 110 is close to an attachment point between the expanded section 110 and the main section of the inner tube 100; and the second attachment point between the second end 320 of the blood blocking member 300 and the outer tube 200 is close to the distal end 210 of the outer tube. The expanded section 110 of the inner tube 100 is designed to enable the blood blocking catheter to maintain a constant outer diameter throughout its whole length. This can avoid damages to the blood blocking member 300 when the blood blocking catheter is passing through a tortuous blood vessel during its delivery in the blood vessel. Moreover, radial distance of the first attachment point relative to the axis of the inner tube 100 substantially equals the distance of the second attachment point relative to the axis of the inner tube 100, which is helpful in maintaining concentricity and avoiding eccentricity, of the blood blocking member 300 during its expansion as well as improving the uniformity of contraction of the blood blocking member 300 to the blood vessel wall to reduce the risk of blood leakage.

Referring to FIG. 8, in one preferred embodiment, the blood blocking member 300 includes an isodiametric section 350. When the blood blocking member 300 is in an expanded configuration, the isodiametric section 350 maintains a constant outer circumferential size along an axis thereof (i.e., the isodiametric section 350 has a cylindrical shape). Optionally, the expanded configuration of the isodiametric section 350 of the blood blocking member 300 is thermally formed to be a tube having a constant diameter. With this arrangement, when the blood blocking member 300 is axially compressed to expand, the isodiametric section 350 expands synchronously throughout its axial length. This results in a shape of the blood blocking member 300 that allows the blood blocking member 300 to better press against the blood vessel wall, which thus provides an even desirable blood blocking performance. It is to be understood that, in some embodiments, the isodiametric section 350 is not limited to expand synchronously throughout its length during the expansion of the blood blocking member 300 and is designed to expand gradually from one end to the other end. In this case, the isodiametric section 350 possesses a sloped outer surface at first (i.e., the isodiametric section 350 has a tapered shape as a whole), and then achieves the configuration having a constant outer circumferential size along its axial direction (i.e., the isodiametric section 350 has a cylindrical shape) until the blood blocking member 300 has expanded fully or to an extent adapted to the inner diameter of the blood vessel wall. However, the present invention is not limited in this regard.

In summary, the flow blocking catheter of the present application comprises an inner tube, an outer tube and a flow blocking member. The flow blocking member has one end attached to an outer circumference of the inner tube and the other end attached to a distal end of the outer tube. The flow blocking member is configured to expand as the outer tube moves toward a distal end of the inner tube and to collapse as the outer tube moves away from the distal end of the inner tube. With this configuration, expansion of the flow blocking member is able to be controlled simply by pushing/retracting the outer or inner tube, which allows to achieve a fast shifting between different configurations, relocatability during a surgical procedure, and a simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or passage of instruments.

The description presented above is merely a few preferred embodiments of the present application and does not limit the protection scope of present application in any sense. Any change and modification made by those of ordinary skill in the art based on the above teachings fall within the protection scope of the appended claims.

What is claimed is:

1. A flow blocking catheter, comprising:
an inner tube;
an outer tube movably sleeved on the exterior of the inner tube; and
a flow blocking member having one end attached to an outer circumference of the inner tube and the other end attached to a distal end of the outer tube, wherein the flow blocking member is configured to expand as the outer tube moves toward a distal end of the inner tube and to collapse as the outer tube moves away from the distal end of the inner tube.

2. The flow blocking catheter of claim 1, wherein the flow blocking member comprises a support frame having opposing ends thereof attached to the inner tube and the outer tube respectively, wherein the support frame is configured to expand when axially compressed and collapse when axially pulled.

3. The flow blocking catheter of claim 2, wherein the flow blocking member further comprises a flow blocking membrane attached to the support frame.

4. The flow blocking catheter of claim 1, wherein the distal end of the inner tube comprises an expanded section that has an outer circumferential size greater than that of rest portion of the inner tube, wherein the flow blocking member is connected to the expanded section at one end and to the distal end of the outer tube at the other end.

5. The flow blocking catheter of claim 4, wherein the outer circumference of the expanded section is sized to fit with an outer circumferential size of the outer tube.

6. The flow blocking catheter of claim 1, wherein the flow blocking member further comprises an isodiametric section that has an equal outer circumferential size along an axial direction of the flow blocking member in an expanded configuration.

7. The flow blocking catheter of claim 1, wherein the flow blocking member further comprises a control valve configured to drive relative movements between the inner and outer tubes.

8. The flow blocking catheter of claim 7, wherein the control valve comprises a control valve body and a control slider configured to be axially slidable, wherein: the control valve body is coupled to a proximal end of the inner tube with the control slider being coupled to a proximal end of the outer tube; or the control valve body is coupled to the proximal end of the outer tube with the control slider being coupled to a proximal end of the inner tube.

9. The flow blocking catheter of claim 1, wherein both or either of the inner tube and the outer tube is a single-layered tube made of macromolecular material.

10. The flow blocking catheter of claim 1, wherein both or either of the inner tube and the outer tube has a structure comprising at least two layers, in which both or either of a first layer and a second layer from inside to outside is made of macromolecular material.

11. The flow blocking catheter of claim 1, wherein both or either of the inner tube and the outer tube has a structure comprising at least two layers, in which a second layer from inside to outside is one or more selected from the group consisting of braided structure, coil, and cut hypotube.

12. The flow blocking catheter of claim 1, wherein each of the inner and outer tubes has a triple-layered structure.

13. The flow blocking catheter of claim 1, wherein the inner tube comprises a first radiopaque ring disposed at the distal end of the inner tube.

14. The flow blocking catheter of claim 13, wherein the inner tube further comprises a second radiopaque ring disposed at a location of the inner tube where the flow blocking member is attached to the inner tube.

15. The flow blocking catheter of claim 13, wherein the outer tube further comprises a third radiopaque ring disposed at a location of the outer tube where the flow blocking member is attached to the outer tube.

16. The flow blocking catheter of claim 1, wherein the flow blocking member comprises at least one selected from the group consisting of mesh structure, open-loop structure and spiral structure, and wherein the flow blocking member is fabricated by braiding, winding or cutting.

17. The flow blocking catheter of claim 16, wherein the mesh structure is braided from 1 to 64 filaments, wherein the filament is at least one selected from the group consisting of regular filament, radiopaque filament and composite filament, the regular filament made of at least one selected from the group consisting of nickel-titanium alloy, cobalt-chromium alloy, stainless steel and macromolecular material, the radiopaque filament made of at least one selected from the group consisting of radiopaque metal, alloy of radiopaque metals and macromolecular material containing a radiopaque agent, the composite filament formed by a radiopaque core filament combined with a regular filament.
